(19) United States
(12) Patent Application Publication
(10) Pub. No.: US 2008/0045839 A1
(43) Pub. Date: Feb. 21, 2008

(54) DETERMINING THE CENTER OF ROTATION OF AN IMPLANT

(76) Inventor: Peter Drumm, Munchen (DE)

Correspondence Address: RENNER, OTTO, BOISSELLE & SKLAR, LLP, Nineteenth Floor, 1621 Euclid Avenue, Cleveland, OH, 44115-2191, US

(21) Appl. No.: 11/840,034

(22) Filed: Aug. 16, 2007

Related U.S. Application Data
(60) Provisional application No. 60/862,076, filed on Oct. 19, 2006.

(30) Foreign Application Priority Data
Aug. 17, 2006 (EP) 06017161

(51) Int. Cl. A61B 6/12; A61B 5/00

(52) U.S. Cl. 600/473; 600/407

(57) ABSTRACT

A device for determining a center of rotation of a medical implant having a spherical portion includes an abutment member, and a plurality of markers arranged in a fixed location relative to the abutment member. The abutment member includes including a first spherical surface for placing the spherical portion of the medical implant, wherein a contour of the first spherical surface at least partially conforms to the spherical portion of the medical implant.

### RELATED APPLICATION DATA

This application claims priority of U.S. Provisional Application No. 60/862,076 filed on Oct. 19, 2006, which is incorporated herein by reference in its entirety.

### FIELD OF THE INVENTION

The present invention relates to implants and, more particularly, to a method, device and system for determining an implant center of rotation.

### BACKGROUND OF THE INVENTION

When performing medical procedures on joints of the body, it is desirable to know a center of rotation of the joint both before and after the procedure. This is particularly true when performing joint replacement procedures, as it is preferable for the artificial joint to duplicate (as close as possible) the geometry of the original joint.

### SUMMARY OF THE INVENTION

An implant center of rotation determination device is described in further detail below. In combination with a detection means (which can detect the markers) and a data processing means (which can process the detection signals), the center of rotation of an implant can be determined.

As noted above, the marker means can be detected by means of the detection means (e.g., a camera or ultrasound detector). The marker means can comprise three markers that are arranged in a fixed and/or predetermined location relative to each other, wherein the marker means may be mechanically connected to one another. The markers can be passive markers, e.g., markers that reflect signals (such as waves and/or radiation) emitted in their direction, or active markers that are the source of the signals (e.g., radiation and/or waves). The signals reflected or emitted from the (active or passive) markers can be detected by the detection means (e.g., the camera). In order to establish a position of the marker means relative to the detection means, the marker means preferably is moved so as to provide the detection means with different views of the marker means. On this basis, the location of the marker means relative to the detection means can be determined in a known way, in particular in a spatial reference system. Reference is made in this respect to DE 196 39 615 A1 and the corresponding U.S. Pat. publication No. 6,351,659, both of which are hereby incorporated by reference in their entirety.

The location of the marker means can be determined by the position of the marker means in a predetermined reference system, wherein the detection means preferably lies within the reference system. The location of the marker means can be determined by the positions of the markers, in particular the center points of the markers, in the reference system. The positions, for example, can be described using Cartesian coordinates or spherical coordinates. The location of one part (e.g., the detection means or marker means) relative to another part (e.g., the marker means or detection means) can be described by spatial angles and/or distances and/or coordinates (in a reference system) and/or vectors, and preferably is calculated from the positions describing the location of the part or parts (e.g., by means of a program running on a computer).

The term “relative location” as used herein or the expression “location of a part A relative to a part B” comprises the concept of the relative positions between the two parts, in particular between the marker means and/or their markers or between the marker means and the detection means. In particular, centers of gravity or center points of the parts can be selected as a punctiform reference point for establishing a position. If the position of one part is known in a reference system, then it is possible, on the basis of the relative location of two parts, to calculate the position of one of the two parts from the position of the other of the two parts. If the marker means comprises only two markers, a start position is preferably known, and the marker system then allows the location of the marker means to be tracked when the marker means is spatially moved.

The marker means preferably comprises at least two markers, and more preferably three or more markers. The dimensions of the markers and the locations of the markers relative to each other are preferably known and in particular available as prior known data of a data processing means. The shape of the markers also is preferably known.

A system for determining an implant center of rotation can comprise the implant center of rotation determination device, a processing means and a detection means. The detection means can detect signals from the at least two markers (e.g., from two or more markers). As stated above, these signals emitted from the markers can be actively emitted by the markers or can be reflected by the markers. In the latter case, a signal transmitting source, for example an infrared light source, can be provided that emits signals (e.g., ultrasound waves or infrared light) toward the passive markers (continuously or in pulses), wherein the passive markers reflect the signals. The data processing means, such as a computer, allows the location of the marker means relative to the detection means to be calculated, in particular the location of the marker means in a reference system in which the detection means lies (e.g., in a reference system that lies in an operating theater).

The data processing means can be designed to calculate and/or determine various parameters. The data processing means can calculate the locations of the marker means based on the detected signals reflected or emitted therefrom. Based on the calculated locations, objects (e.g., a body structure or instruments) to which the marker means are attached can be calibrated. This means that the relative locations at least between parts of the object and the marker means attached to the object can be known and/or stored or input in the data processing means, such that indicating signals that describe the locations of the objects can be determined based on the locations of the marker means. The locations of the marker means can be calculated relative to the detection means, e.g., in a reference system in which the detection means lies. The locations also can be calculated in another reference system, e.g., in a reference system in which the patient lies and/or in which one of the marker means lies.

As stated above, the implant center of rotation determination device comprises an abutment member that is to be placed onto an implant. This implant can be spherical, e.g., a part of the surface of the implant that corresponds to the surface of a sphere. To determine the spherical center point of the implant, a spherically configured surface of the abutment member can be placed onto the spherical surface of the implant, wherein the spherical surface of the implant can be convex and the spherical surface of the abutment member can be concave. Alternatively, the spherical surface of the implant can be concave and the spherical surface of the abutment member can be convex. In both cases, the surface of the abutment member is complementary to the implant. The abutment member preferably is configured and/or selected such that the spherical surface of the abutment member solidly abuts the spherical surface of the implant, e.g., the two surfaces contact each other.

When using the implant center of rotation determination device, markers of the implant center of rotation determination device can be stationary relative to the abutment member, and their location relative to the abutment member preferably is known. The location of the markers relative to the spherical center point of the spherical surface of the abutment member also is preferably known. Thus, if the location of the markers is determined, the location of the spherical center point of the abutment member can be calculated. Since the spherical surface of the abutment member solidly abuts the spherical surface of the implant, the two spherical center points correspond to each other. Thus, if the location of the markers is known in a reference system (the operating theater can be the reference system in which the camera lies), then the spherical center point of the implant, and therefore the location of the center of rotation, also can be determined from the location of the markers and the location of the markers relative to the spherical center point of the abutment member.

The markers can form a marker means and/or can be constituents of a marker means, wherein the marker means can be detachably attached to the abutment member. This enables differently configured marker means (e.g., reference stars) to be attached to the abutment member and to facilitate cleaning the abutment member.

As already stated above, the abutment member can be placed solidly onto the implant. To accomplish this, the curvature of the spherical surface of the abutment member should correspond to the curvature of the spherical surface of the implant and/or at least correspond by 90% or more. To this end, different implant center of rotation determination devices can be provided that are available to an operator (e.g., a surgeon) and comprise abutment members having spherical surfaces of different curvatures. The implant center of rotation determination device can be configured such that the abutment member is configured in two parts, wherein one part (e.g., the abutment area member) can be configured to be exchangeable. The abutment member can comprise a carrier and an abutment area member, wherein different abutment area members can be connected, in particular detachably connected, to the carrier. The abutment area members can be exchanged, wherein different abutment area members comprise spherical surfaces of different curvatures. The carrier can be configured such that the markers are attached thereto or a marker means can be attached to the carrier, preferably detachably attached to the carrier.

The abutment member can be configured such that abutment area members having optionally convex or concave spherical surfaces and/or different embodiments (e.g., different curvature radii of the spherical abutment area) can be attached to the abutment member, wherein a concave abutment area member may be designed having a cup-shape and comprise a spherical recess. An abutment area member having a convex spherical surface can comprise an attaching member that preferably is detachably attached to the carrier. A distancing member (e.g., an appendage), for example a rod or connecting piece, also can be provided that connects a contact area member, e.g., a head having a spherical surface, to the abutment member thereby creating a distance between the carrier and the contact area member (head). The contact area member can be brought into contact with the concave spherical implant by the operator.

An implant center of rotation determination system comprises the implant center of rotation determination device described herein and also a detection means and a data processing means. The detection means can detect signals from the markers. The detected signals can be processed by the data processing means, which, via the detected signals, can calculate the location of the spherical center point of the abutment member, and therefore the center of rotation of the implant.

The data processing means can determine the location of the spherical center point in different ways. In accordance with a first example, the location of the spherical center point relative to the markers (and to the marker means) can be known, wherein “known” means that the data corresponding to the relative location are stored in the data processing means and/or input into the data processing means. The data processing means then can process the data together with the detected location of the markers to determine the location of the spherical center point of the abutment member and therefore of the implant. To this end, the implant center of rotation determination device may be placed solidly onto the spherical surface of the implant, while the signals from the markers can be detected and then further processed to determine the location of the implant center of rotation determination device.

The relative location between the spherical center point of the abutment member and the markers may be unknown. In this case, the implant center of rotation determination device can be moved while it is in solid contact with the spherical surface of the implant. The trajectories of the movement preferably span a spherical area (e.g., there should be a pivoting movement about two different axes), wherein at least three different locations of the markers or marker means are preferably detected. Numerical methods can be used to determine whether these different locations lie on a spherical surface and if so, the center point of the sphere corresponding to this spherical surface as well as the radius of the sphere. Determining the location of the center point also determines the spherical center point of the abutment member and also the spherical center point of the spherical surface of the implant.

### BRIEF DESCRIPTION OF THE DRAWINGS

The forgoing and other features of the invention are hereinafter discussed with reference to the drawing.

FIG. 1 illustrates an exemplary device for determining an implant center of rotation in accordance with the invention, wherein the device can be used to determine a spherical center point of implants having a convex spherical surface.

FIG. 2 is a schematic sectional view of the device of FIG. 1.

FIG. 3 illustrates an exemplary application of the device of FIG. 1 in accordance with the invention.

FIG. 4 illustrates another exemplary device for determining an implant center of rotation in accordance with the invention, wherein the device can be used to determine a spherical center point of implants having a concave spherical surface.

FIG. 5 illustrates an exemplary system for determining an implant center of rotation in accordance with the invention, wherein the system uses the implant center of rotation determination device in accordance with FIG. 4.

FIG. 6 is a block diagram of an exemplary computer system that can be used to implement the method described herein.

### DETAILED DESCRIPTION

The present invention enables a leg length difference in a hip endoprosthesis, an anteversion of the femur, medial-lateral and/or cranial-caudal shift after implantation to be calculated.

The term “spherical surface” as used herein designates a convex or concave spherical surface, wherein the surface at least partially corresponds to a sphere. The center point of this sphere is a spherical center point of the spherical surface. The spherical center point of the spherical surface of an implant corresponds to a center of rotation of the implant.

Markers described herein can be directly attached to an abutment member or can form a marker means separate from the abutment member. A marker means can comprises markers and other elements, such as, for example, arms that connect the markers to each other in a fixed or predetermined location.

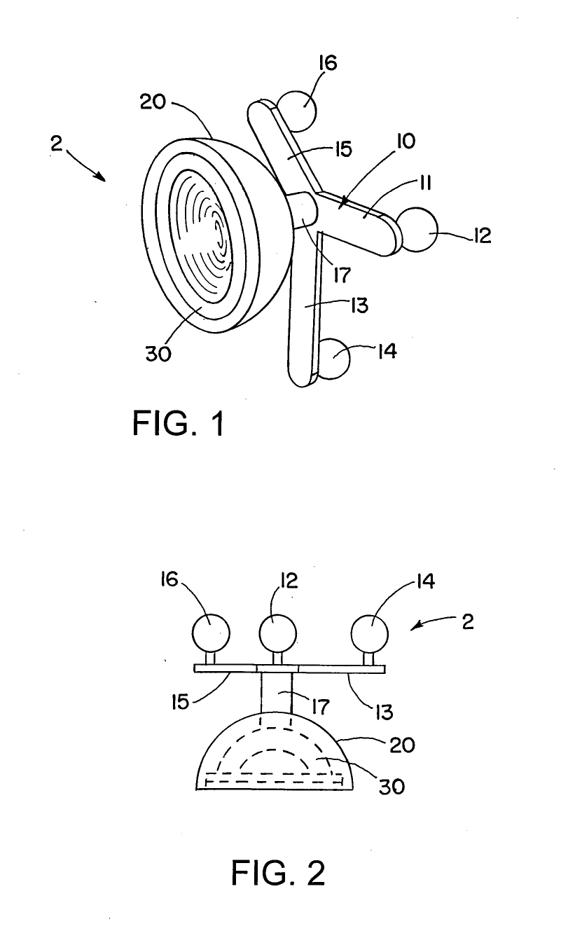

FIG. 1 illustrates an exemplary device for determining an implant center of rotation 2 (referred to hereinafter as ICORD 2). The ICORD 2 comprises markers 12, 14 and 16 that can be coupled to a carrier 20 via arms 11, 13 and 15 and connecting piece 17 (e.g., coupled in a fixed or rigid manner). The connecting piece 17 connects the arms 11, 13 and 15 to a carrier 20. The arms 11, 13 and 15 ensure that the markers 12, 14 and 16 are provided in a particular, characteristic spatial arrangement relative to each other. The markers 12, 14, 16, together with the arms 11, 13 and 15 and the connecting piece 17, form a marker means or marker device. Alternatively, the markers can be directly attached to the carrier 20 or, if a carrier is not provided, directly attached to an abutment area member 30, without the arms 11, 13 and 15 or connecting piece 17 being provided. The marker means 10 can be detached from the carrier 20 and re-connected thereto in a fixed or stationary manner. Thus, depending on the application, different marker means can be used.

The abutment area member 30 can be provided within the carrier 20. The carrier 20, however, is not compulsory. If a carrier 20 is not provided, then as noted above, the markers can be directly attached to the abutment area member 30. The abutment area member 30 forms an area for placing a spherical body, in particular a spherical implant.

FIG. 2 is a schematic lateral view of the ICORD 2 of FIG. 1. Identical parts are again designated by identical reference signs. The spherical recess formed by the abutment area member 30 can be seen here in section (the abutment area member 30 is designed concavely).

As can be seen in FIG. 2, the carrier 20 is designed as a spherical cup and likewise comprises a concave, spherical inner area which the outer area of the abutment area member 30 solidly abuts. The abutment area member 30 can be connected to the carrier 20 as a press fit. Alternatively or additionally, a plug connection, for example, can be provided, in which appendages and recesses in the abutment area member 30 and the carrier 20 interlock to ensure a stationary connection between the abutment area member 30 and the carrier 20. The connection can be designed to be detachable and, for example, can be a latch connection.

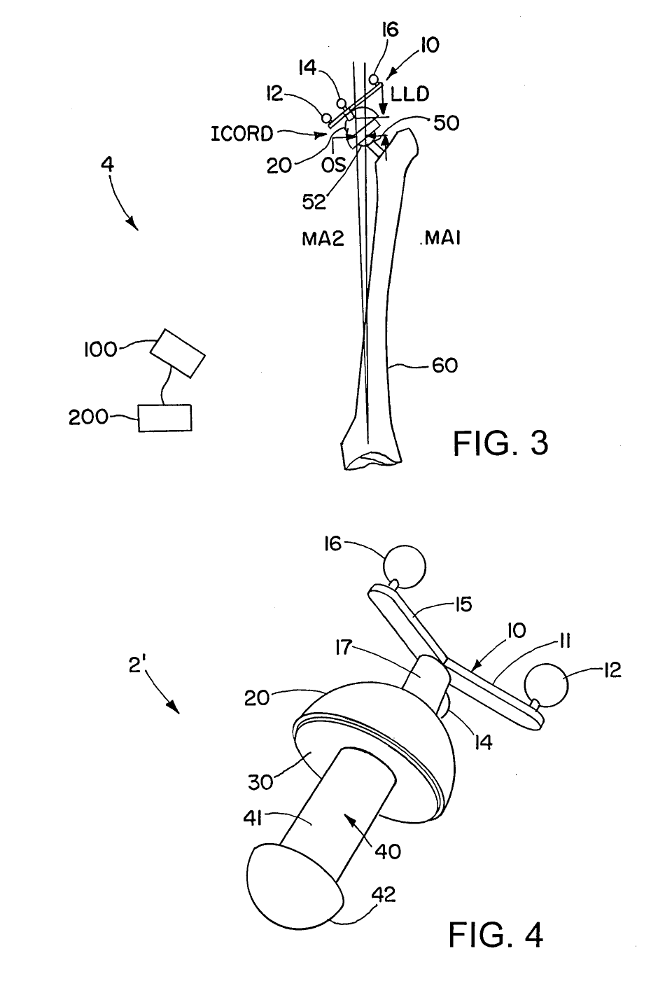

FIG. 3 shows an exemplary application for the ICORD 2, wherein a head of a upper leg bone 60 is resected and an implant 50 is inserted into the upper leg bone, as is performed in the case of a so-called femur neck endoprosthesis. The ICORD 2 is placed over a spherical head 52 of the implant 50, such that the convex spherical head 52 of the implant 50 solidly abuts the concave spherical inner area of the ICORD 2. Signals, such as infrared light that is emitted continuously or in pulses by a transmitter, originate from or are reflected by the markers 12, 14 and 16 and detected by the camera 100. The transmitter, for example, can be accommodated in the camera 100. The detected signals can be further processed by the data processing means 200. The ICORD 2, for example, is a device in which the location of the spherical center point of the abutment area member 30 relative to the marker means 10, and therefore relative to the markers 12, 14 and 16, is known or can be determined. Since the ICORD 2 solidly abuts the implant head 52, the location of the spherical center point can be determined by the data processing means 200 from the location of the marker means as detected by the camera 100. Since the implant head 52 solidly abuts the abutment area member 30, the spherical center point of the abutment area member 30 corresponds to the spherical center point of the implant head 52.

An implant center of rotation determination system 4 (in the following, ICORS 4 for short) can determine the spherical center point of the implant head 52, wherein said system comprises the detection means 100 and the data processing means 200 in addition to the ICORD 2. Before the operation, the center of rotation can be determined by determining the point of rotation of the joint (in this example the upper leg bone (femur)). To this end, different positions of the hip bone and the hip cavity relative to each other can be determined, for example, by attaching reference stars to the registered hip bone and hip cavity and then moving the hip bone relative to the hip cavity. Additional images can be produced in this case by fluoroscopy, as described in EP 1 611 863, the contents of which is hereby incorporated by reference in its entirety.

The pre-operatively determined location of the center of rotation can be read into the data processing means 200, such that the pre-operative location of the center of rotation can be compared with the post-operative location of the center of rotation, as determined by means of the ICORS 4.

FIG. 3 also shows an offset OS resulting from a medial-lateral shift in the center of rotation. Additionally, the leg length difference LLD resulting from a cranial-caudal shift in the center of rotation is shown. It is thus possible, with the aid of the ICORS 4, to determine whether the center of rotation has been changed by the implant (i.e. by the operation) or remains the same. Other implants or implant heads can then be used to achieve the best possible correspondence between the pre-operative center of rotation and the post-operative center of rotation.

The lines indicated longitudinally in FIG. 3 are the so-called mechanical axes MA1 and MA2. The mechanical axis of an upper leg bone (femur) can be defined by the center point of the axis of the epicondyle of the femur and by the center of rotation. The epicondyle is the protrusion of bone attached to a condyle for muscular origins or attachments. Two epicondyles (the medial epicondyle and the lateral epicondyle) are situated at the distal end of the femur. The line connecting the two epicondyles constitutes the epicondylar axis. The center point of the connecting line between the two epicondyles (medial epicondyle, lateral epicondyle) defines the distal end point of the mechanical femur axis.

The ICORS 4 enables the mechanical axis MA1 after the operation to be determined and compared with the mechanical axis MA1 before the operation. The ICORS 4 also enables the anteversion of the femur to be determined. To this end, an angle between the epicondylar axis and a second axis can be determined. The second axis can be defined by the center of rotation and the piriformis fossa point. The piriformis fossa point can be determined by registering the upper leg bone. The data representing the registered bone can be stored in the data processing means 200. A reference star or the like can be attached to the bone, the location of which relative to the bone is known, such that the location of the bone and therefore also the location of the epicondylar axis and the piriformis fossa point relative to the ICORD 2 can be determined from the detected reference star.

FIG. 4 shows a second embodiment of an ICORD 2′. In this embodiment, a contact area member 42 for interfacing with an implant is designed as a convex head. This is different from the embodiment shown in FIG. 1, wherein the abutment area is designed concavely. The ICORD 2′ in accordance with FIG. 4 can be used to determine the center of rotation of a spherically concave implant. As shown in FIG. 4, the ICORD 2′ preferably uses components of the ICORD 2 shown in FIG. 1. Identical parts are again provided with identical reference signs. Thus, the marker means 10, the carrier 20 and the abutment area member 30 are again provided. In this case, the abutment area member 30 serves as an attaching member that can be connected in a stationary or fixed manner and preferably detachably connected to the carrier 20. In the attaching member, i.e., in its concave recess, an insertion piece 40 can be inserted and connected thereto, preferably in fixed and/or detachable manner. Due to the modular design of the ICORD 2′, it can be used to measure both a spherically convex implant and a spherically concave implant. The insertion piece 40 allows an ICORD 2′ which is suitable for measuring a spherically convex implant to be modified into an ICORD 2′ which is suitable for measuring a spherically concave implant. To this end, the insertion piece 40 has a spherically convex head 42 which can be inserted into a spherically concave implant and solidly placed onto the implant. By using insertion pieces of different sizes, in particular different head configurations (different head diameters), it is possible to determine the center of rotation of many different implants having different spherical radii (curvature radii) and in particular to determine their spherical center point. The same applies to the ICORD 2 in accordance with FIG. 1, in which many different convex implants can be determined, in particular their centers of rotation, by using different abutment area members having different spherical radii of the spherical recess.

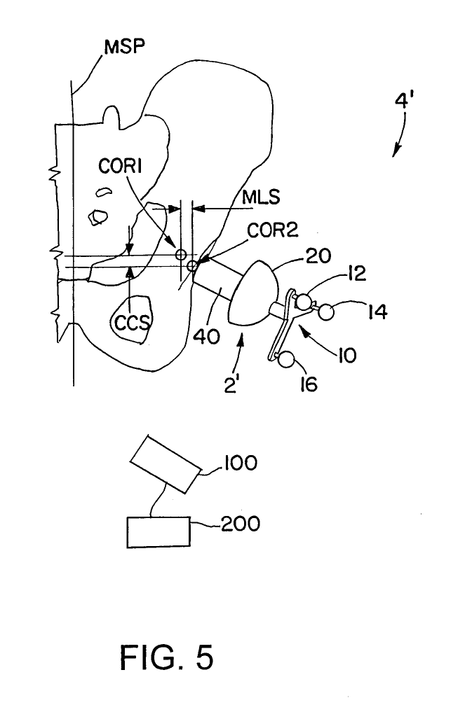

FIG. 5 shows the ICORD 2′ in accordance with FIG. 4 being used to determine the spherical center point of an implant having a concave, spherical recess. In FIG. 5, a hip cavity implant (not visible) has been implanted into the hip. The hip cavity implant has a spherical center point COR2 and is designed cup-shaped. The head 42 (not visible) of the ICORD 2′ of FIG. 4 is inserted into the hip cavity implant, such that the insertion piece 40 protrudes out of the hip. In the case shown in FIG. 5, the difference in location between the pre-operative center of rotation COR1 and the (pre-operative) center of rotation COR2 after the operation also can be determined. In particular, the cranial-caudal shift CCS and the medial-lateral shift MLS can be determined. The middle sagittal plane of the hip bone is designated by MSP.

The implant center of rotation determination system ICORS 4′ of FIG. 5 likewise comprises a detection means 100, which can be designed as a camera, and a data processing means 200.

Similar to FIG. 3, there are two alternatives for determining the post-operative center COR2. In accordance with one alternative, the geometry of the head 42 of the insertion piece 40 is known, in particular the location of the spherical center point of the head 42 relative to the marker means 10 is known. In this application, “known” means that corresponding data about the relative location are available in the data processing means 200, e.g., stored in the data processing means 200 or have been input into the data processing means.

In accordance with another embodiment, the location of the spherical center point of the head 42 relative to the marker means is not known. In this case, pivoting movements can be performed using the ICORD, wherein care must be taken that the head 42 solidly abuts the concave, spherical surface of the implant. The spatial pivoting movements can be detected by the detection means 100. The pivoting movements can be movements about the point of rotation COR2. Based on the assumption that the markers 12, 14 and 16 move on a spherical surface, the center point of the spherical surface thus can be determined from the detected signals.

If the center of rotation COR2 is known, it can be compared with the pre-operatively determined center of rotation COR1. The pre-operative center of rotation, for example, can be determined exactly as in the case described in FIG. 3, e.g., by moving the upper leg bone.

If the pre-operative center of rotation COR1 is known, both the cranial-caudal shift and the medial-lateral shift can be determined by comparing the pre-operative center of rotation COR1 with the determined location of the post-operative center of rotation COR2.

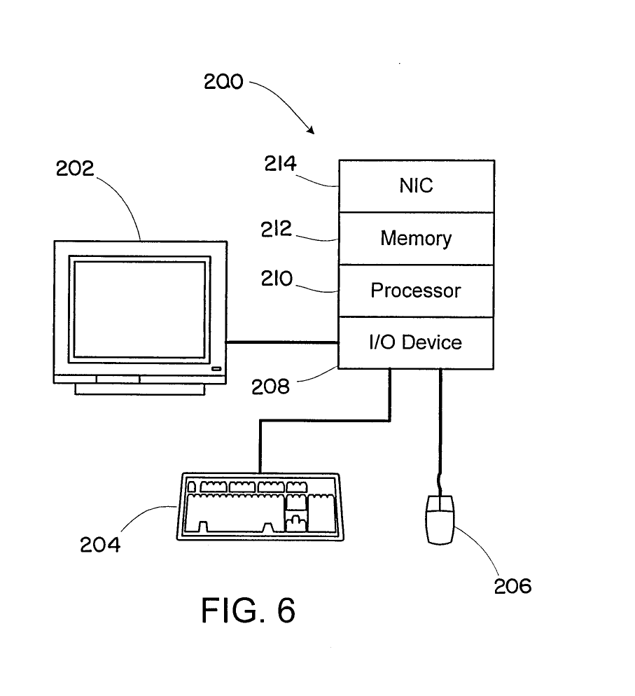

Moving now to FIG. 6 there is shown a block diagram of an exemplary data processing means 200 embodied as a computer that may be used to implement one or more of the methods described herein. The computer 200 may include a display 202 for viewing system information, and a keyboard 204 and pointing device 206 for data entry, screen navigation, etc. A computer mouse or other device that points to or otherwise identifies a location, action, etc., e.g., by a point and click method or some other method, are examples of a pointing device 206. Alternatively, a touch screen (not shown) may be used in place of the keyboard 204 and pointing device 206. The display 202, keyboard 204 and mouse 206 communicate with a processor via an input/output device 208, such as a video card and/or serial port (e.g., a USB port or the like).

A processor 210, such as an AMD Athlon 64® processor or an Intel Pentium IV® processor, combined with a memory 212 execute programs to perform various functions, such as data entry, numerical calculations, screen display, system setup, etc. The memory 212 may comprise several devices, including volatile and non-volatile memory components. Accordingly, the memory 212 may include, for example, random access memory (RAM), read-only memory (ROM), hard disks, floppy disks, optical disks (e.g., CDs and DVDs), tapes, flash devices and/or other memory components, plus associated drives, players and/or readers for the memory devices. The processor 210 and the memory 212 are coupled using a local interface (not shown). The local interface may be, for example, a data bus with accompanying control bus, a network, or other subsystem.

The memory may form part of a storage medium for storing information, such as application data, screen information, programs, etc., part of which may be in the form of a database. The storage medium may be a hard drive, for example, or any other storage means that can retain data, including other magnetic and/or optical storage devices. A network interface card (NIC) 214 allows the computer 200 to communicate with other devices.

A person having ordinary skill in the art of computer programming and applications of programming for computer systems would be able in view of the description provided herein to program a computer system 200 to operate and to carry out the functions described herein. Accordingly, details as to the specific programming code have been omitted for the sake of brevity. Also, while software in the memory 212 or in some other memory of the computer and/or server may be used to allow the system to carry out the functions and features described herein in accordance with the preferred embodiment of the invention, such functions and features also could be carried out via dedicated hardware, firmware, software, or combinations thereof, without departing from the scope of the invention.

Computer program elements of the invention may be embodied in hardware and/or in software (including firmware, resident software, micro-code, etc.). The invention may take the form of a computer program product, which can be embodied by a computer-usable or computer-readable storage medium having computer-usable or computer-readable program instructions, “code” or a “computer program” embodied in the medium for use by or in connection with the instruction execution system. In the context of this document, a computer-usable or computer-readable medium may be any medium that can contain, store, communicate, propagate, or transport the program for use by or in connection with the instruction execution system, apparatus, or device. The computer-usable or computer-readable medium may be, for example but not limited to, an electronic, magnetic, optical, electromagnetic, infrared, or semiconductor system, apparatus, device, or propagation medium such as the Internet. Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner. The computer program product and any software and hardware described herein form the various means for carrying out the functions of the invention in the example embodiments.

Although the invention has been shown and described with respect to a certain preferred embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

What is claimed is:

1. A device for determining a center of rotation of a medical implant, said medical implant including a spherical portion, comprising:
an abutment member including a first spherical surface for placing the spherical portion of the medical implant, wherein a contour of the first spherical surface at least partially conforms to the spherical portion of the medical implant; and
a plurality of markers arranged in a fixed location relative to the abutment member.

2. The device according to claim 1, wherein the first spherical surface of the abutment member is a convex surface.

3. The device according to claim 1, wherein the first spherical surface of the abutment member is a concave surface.

4. The device according to claim 1, wherein the first spherical surface conforms to at least 90 percent of the spherical portion of the medical implant.

5. The device according to claim 1, wherein the abutment member comprises:
a carrier; and
an abutment area member detachably coupled to the carrier, wherein the abutment area member includes the first spherical surface.

6. The device according to claim 5, wherein the abutment area member includes a second spherical surface, and the carrier includes a third spherical surface, wherein a contour of the second spherical surface conforms to a contour of the third spherical surface.

7. The device according to claim 5, wherein the carrier is coupled to a marker device, said marker device including the plurality of markers.

8. The device according to claim 5, wherein the abutment area member comprises a recess with a concave spherical area.

9. The device according to claim 5, wherein the abutment area member comprises an appendage with a head having a convex spherical surface.

10. A system for determining a center of rotation of a medical implant, comprising:
the device according to claim 1;
a detection device for detecting a location of the plurality of markers; and
a data processing device communicatively coupled to the detection device, said data processing device operative to determine a location of a center point of the first spherical surface of the abutment member based on the detected location of the plurality of markers, and at least one of
a) a known location of the spherical center point of the first spherical surface of the abutment member relative to the markers; or
b) a plurality of the detected locations of the markers, wherein the plurality of detected locations represent different locations of the device relative to the spherical center point of the first spherical surface when the first spherical surface is abutting the implant.

11. The system according to claim 10, wherein the detection device comprises an optical detection device.

12. The system according to claim 11, wherein the optical detection device comprises an infrared camera.

13. A method for determining a center of rotation of a medical implant using a device having an abutment member for placing a spherical portion of the medical implant, and a plurality of markers arranged in a fixed location relative to the abutment member, the method comprising:
selecting a spherical surface of the abutment member that has a radius corresponding to a radius of the spherical portion of the implant;
placing the spherical portion of the implant onto the spherical surface of the abutment member;
determining at least one location of the plurality of markers; and
determining a spherical center point of the implant based on at least one of
a) a plurality of determined locations of the plurality of markers, or
b) at least one determined location of the plurality of markers and on a known location of a spherical center point of the spherical surface relative to the markers.

14. The method according to claim 8, wherein determining at least one location of the markers includes using an optical detection device to determine the location of the markers.
